(19) United States
(12) Patent Application Publication
(10) Pub. No.: US 2009/0022765 A1
(43) Pub. Date: Jan. 22, 2009

(54) COSMETIC PIGMENT COMPOSITION CONTAINING GOLD OR SILVER NANO-PARTICLES

(75) Inventors: Bong Hyun Chung, Daejeon (KR); Yong Taik Lim, Daejeon (KR); Jin Kyeong Kim, Daejeon (KR); Jin Young Jeong, Daejeon (KR); Tai Hwan Ha, Seoul (KR)

Correspondence Address: INTELLECTUAL PROPERTY / TECHNOLOGY LAW, PO BOX 14329, RESEARCH TRIANGLE PARK, NC, 27709, US

(73) Assignee: Korea Research Institute of Bioscience and Biotechnology, Daejeon (KR)

(21) Appl. No.: 11/995,847

(22) PCT Filed: Feb. 13, 2006
(86) PCT No.: PCT/KR06/00493

§ 371 Date: Apr. 16, 2008

(30) Foreign Application Priority Data
Jul. 18, 2005 (KR) 10-2005-0064778

(51) Int. Cl. A61K 8/19; A61K 8/00; A61Q 1/00; A61Q 1/04; A61Q 1/10

(52) U.S. Cl. 424/401; 424/63; 424/618; 424/649; 424/64; 424/70.7

(57) ABSTRACT

The present invention relates to a cosmetic pigment composition exhibiting colors in the visible region, which comprises an effective amount of nanoparticles or a mixture of two or more nanoparticles selected from the group consisting of (a) gold nanoparticles exhibiting red color; (b) silver nanoparticles exhibiting yellow color; (c) gold-silver alloy nanoparticles exhibiting flame color; and (d) gold nanoparticles exhibiting blue color, and a color cosmetic composition and a color lotion comprising the pigment composition. According to the present invention, it is possible to prepare pigments exhibiting various colors in the visible region using gold or silver nanoparticles, and a cosmetic pigment composition which can exhibit various colors by mixing the pigments in various compositional ratios, in which precipitation or agglomeration of particles does not occur, and whose color can be maintained for a long time. Also, since the pigment of the present invention is not harmful to the human body unlike conventional metal pigments, and contains gold or silver that is beneficial to health, the pigment can be used in various applications as functional raw materials.

### TECHNICAL FIELD

The present invention relates to a cosmetic pigment composition containing gold or silver nanoparticles, and more particularly to a cosmetic pigment composition exhibiting the colors of the visible light spectrum, which contains an effective amount of nanoparticles or a mixture of two or more nanoparticles selected from the group consisting of: (a) gold nanoparticles exhibiting red color; (b) silver nanoparticles exhibiting yellow color; (c) gold-silver alloy nanoparticles exhibiting orange color; and (d) gold nanoparticles exhibiting blue color.

### BACKGROUND ART

The main materials of pigments which are mainly used in most cosmetics, including lipsticks, include about 90 kinds of tar pigments separated and synthesized from petroleum. Also, metal compounds are used as pigments to develop colors, and examples thereof include heavy metal compounds such as lead, cadmium, iron oxide. These pigments show excellent color development, thus leading to clear colors, and some of these pigments have good thermal resistance and light resistance, but are insoluble in water and soluble only in some solvents. Also, they are generally present in a dispersed form, but are difficult to maintain in the form of a stable dispersion for a long period of time due to the cohesion between pigment particles or the settlement of the pigment particles.

In addition, these pigments are substances harmful to the human body, which can cause side effects such as skin diseases. To improve the problem with the use of the above-descried cosmetic pigments, various studies on cosmetic pigments have been conducted. For example, Korean Patent Publication 10-1997-009773 discloses a cosmetic pigment containing a large amount of saponarin extracted from the rose of Sharon. This pigment contains a physiologically active material effective in the human body and can substitute for metal compounds which are pigments in the prior art, but has a problem in that, when it is used in cosmetics, it should still use organic material or synthetic resin particles in order to prevent the greasiness of skin.

Meanwhile, Korean Patent Publication 10-2005-0030398 discloses a make-up cosmetic composition capable of effectively inhibiting greasiness caused by sebum secretion, which contains gold nanoparticle-containing silica fine particles. However, the gold nanoparticles used herein are limited to 20-50 nm wavelength in the wavelength range exhibiting red color, and it is difficult for the gold nanoparticles to exhibit various colors.

Nanometer-sized materials have physical properties different from micrometer- or meter-sized materials, and show very unique optical properties, particularly in the case of metal materials. For example, in case of gold, if its size becomes several hundreds nanometers of spherical form, it begins to exhibit red color not yellow color that is observed generally, and in sizes similar thereto, silver nanoparticles show a yellow color, not a gray silver color. These phenomena are caused by Surface Plasmon Resonance Effect that strongly absorbs light of a certain wavelength and reflects the rest according to a size and a shape, which occurs on a surface of metal. Because of the effect, one can recognize only reflected wavelength. Namely, since gold nanoparticles having a size of nanometers absorb wavelength corresponding to green color and reflect the remaining wavelength, the gold nanoparticles exhibit red color. Additionally, nanoparticles that absorb wavelengths different from each other can be prepared by varying the size and the shape of gold or silver particles.

Meanwhile, the effects of gold have been well known from ancient times. For example, Roman naturalist Plinius recorded in the 1st century BC that gold had an effect to treat skin ulcer, and a liquid medicine containing gold powder was used for anti-aging in middle ages. Meanwhile, modern medical science found that gold have the ability to inhibit proliferation of bacillus and treat asthma. Also, it was found that gold had the ability to alleviate the pain of rheumatoid arthritis in 1927 in Europe, and fine gold particles have been used in internal medicine and ophthalmology. Also, Korean medical books, including Dongeuibogam, disclose that gold has various effects on tranquilization, neutralization of poison, skin clarification, treatment of arthritis, treatment of neuralgia or the like. As described above, the effects of gold are well known.

Additionally, it was found that silver can disinfect about 650 kinds of bacteria, almost all fungi, bacillus and virus, and can stop the function of enzymes that are necessary for proliferation procedure of bacteria. Also, silver has various effects on treating burns, promotion of bone growth, regeneration and activation of cell, an increase of immunogenecity, hematopoiesis assisting the circulation of lymph fluids, removing malodor by disinfections of various bacteria that are the cause of malodor and neutralization of ammonia and degenerated proteins, and detoxification of toxins in human body including alcohol dehydrogenation or the like.

However, in the prior arts, it is not yet reported that gold or silver nanoparticles are used to develop colors in addition to red color or yellow color. Further, since gold and silver have no toxicity unlike a conventional pigment, and have strong disinfecting abilities and high stabilities, they are valuable substances which are used as high-grade raw materials in food and cosmetics.

In prior arts, nanoparticles have been used as carriers to deliver substances (natural substances or synthetic substances) having effects including whitening, sunless tanning, anti-aging, treatment of skin diseases (acne, seborrhea and seborrheic dermatitis) or the like, to the skin (US Patent Publication 2004/0166069; US Patent Publication 2005/0175556; US Patent Publication 2004/0161435; and US Patent Publication 2005/0238597). However, it has not been yet reported that metal nanoparticles (gold or silver nanoparticles) exhibiting colors other than red color or yellow color are used as pigments for cosmetic composition.

Accordingly, the present inventors prepared gold nanoparticles exhibiting red color, silver nanoparticles exhibiting yellow color and gold nanoparticles exhibiting blue color, and mixed these nanoparticles with each other at a suitable ratio and, as a result, found that a cosmetic pigment composition exhibiting various colors could be prepared, thereby completing the present invention.

### SUMMARY OF INVENTION

Accordingly, the present invention has been made to solve the above-mentioned problems occurring in the prior art, and it is an object of the present invention to provide a cosmetic pigment composition comprising gold or silver nanoparticles, which is harmless to the human body and can show various colors.

It is another object of the present invention to provide lotions or color cosmetics comprising said cosmetic pigment composition.

To accomplish the above objects, according to the present invention, there is provided a cosmetic pigment composition exhibiting the colors of the visible light spectrum, the composition comprising an effective amount of nanoparticles or a mixture of two or more nanoparticles selected from the group consisting of: (a) gold nanoparticles exhibiting red color; (b) silver nanoparticles exhibiting yellow color; (c) gold-silver alloy nanoparticles exhibiting orange color; and (d) gold nanoparticles exhibiting blue color.

In the present invention, the nanoparticles can be in the form of shapes selected from the group consisting of nanospheres, nanorods, nanoshells, nanocubes and nanoprisms. Preferably, the gold nanoparticles exhibiting red color and the silver nanoparticles exhibiting yellow colors can be in the form of nanospheres, and the gold nanoparticles exhibiting blue color can be in the form of shapes selected from the group consisting of nanoshells, nanorods, nanocubes and nanoprisms.

In the cosmetic pigment composition, the nanoparticle mixture is preferably selected from the group consisting of: (a) a mixture in which gold nanoparticles exhibiting red color and silver nanoparticles exhibiting yellow color are mixed with each other at various ratios; (b) a mixture in which silver nanoparticles exhibiting yellow color and gold nanoparticles exhibiting blue color are mixed with each other at various ratios; (c) a mixture in which gold nanoparticles exhibiting red color and gold nanoparticles exhibiting blue color are mixed with each other at various ratios; (d) a mixture in which nanoparticles exhibiting three colors of red color, yellow color and blue color are mixed with each other at various ratios.

In the present invention, the gold nanoparticles exhibiting red color is preferably prepared through a method comprising the steps of:
(a) refluxing a solution of HAuCl₄ at about 100° C.;
(b) adding a reducing agent to the refluxed solution, and then heating the solution so as to react with the reducing agent; and
(c) cooling the reaction solution to room temperature and filtering the cooled solution.

However, the method of preparing the gold nanoparticles exhibiting red color is not limited to the above-described method.

Also, the silver nanoparticles exhibiting yellow color is preferably prepared through a method comprising the steps of:
(a) refluxing a solution of AgNO₃ at about 100° C.;
(b) adding a reducing agent to the refluxed solution, and heating the solution so as to react with the reducing agent; and
(c) cooling the reaction solution to room temperature and filtering the cooled solution.

However, the method of preparing the silver nanoparticles exhibiting yellow color is not limited to the above-described method.

Also, the gold nanoparticles exhibiting blue color is preferably prepared through a method comprising the steps of:
(a) mixing AgNO₃, PVP (polyvinylpyrrolidone) and EG (ethylene glycol), heating and reacting the mixture at about 120° C., and then cooling the reaction solution;
(b) adding a reducing agent to the cooled solution, heating the solution at about 100° C., and then allowing the heated solution to react with gold ion (HAuCl₄), which is added in portions; and
(c) cooling the reaction solution to room temperature and filtering the cooled solution.

However, the method of preparing the gold nanoparticles exhibiting blue color is not limited to the above-described method.

Meanwhile, the gold-silver alloy nanoparticles exhibiting orange color is preferably prepared through a method comprising the steps of:
(a) heating a reducing agent solution, adding a mixture of gold ion (HAuCl₄) and silver ion (AgNO₃) to the heated reducing agent solution, and allowing the mixture solution to react; and
(b) cooling the reaction solution to room temperature and filtering the cooled solution.
However, the method of preparing the gold-silver alloy nanoparticles exhibiting orange color is not limited to the above-described method.

Furthermore, the present invention provides color cosmetics comprising said pigment composition. The color cosmetics preferably have a formulation selected from the group consisting of twin-cake, emulsion foundation, make-up base, skin cover, eye shadow, face powder, a lipstick, mascara, nail-lacquer and eyebrow pencil.

Additionally, the present invention provides a composition for color lotions comprising said pigment composition. The composition for color lotions preferably have a formulation selected from the group consisting of skin softeners, skin lotions and essences.

Other features and embodiments of the present invention will be more fully apparent from the following detailed description and appended claims.

### BRIEF DESCRIPTION OF DRAWINGS

FIG. 1 shows pigment composition exhibiting green, violet and orange colors, prepared by mixing spherical gold nanoparticles exhibiting red color, spherical silver nanoparticles exhibiting yellow color and gold nanoshell particles exhibiting blue color with each other in various ratios.

FIG. 2 shows color lotions exhibiting various colors, prepared by adding pure gold or silver nanoparticles or a mixture thereof to conventional lotions.

FIG. 3 shows a nano-gold lipstick prepared by adding a red color of spherical gold nanoparticle pigment composition to lipstick raw materials.

### DETAILED DESCRIPTION OF THE INVENTION AND PREFERRED EMBODIMENTS

The present invention relates to a non-toxic and stable cosmetic pigment composition comprising gold or silver nanoparticles which can be used to develop various colors in lotions such as skin lotions or color cosmetics such as lipsticks, as well as color cosmetics and color cosmetic compositions which comprise said pigment composition.

Gold or silver nanoparticles according to the present invention can have the shape of nanospheres, nanoshells, or nanorods. However, spherical gold nanoparticles and silver nanoparticles generally exhibit red color and yellow color, respectively.

In the present invention, various colors in the visible light spectrum can be developed using gold nanoshell particles exhibiting blue color in addition to the gold and silver nanoparticles exhibiting red color and yellow color, respectively. Namely, in addition to red color, yellow color and blue color, orange colors can be developed by mixing red color with yellow color, green colors can be developed by mixing yellow color with blue color, and violet colors can be developed by mixing red color and blue color. In the present invention, orange color can also be developed by preparing nano gold-silver alloy colloids.

To prepare colloidal nanoparticles, an aqueous solution containing gold ion or silver ion is heated, and then a given concentration of a reducing agent is added to the heated solution. In this case, various sizes of particles (generally, spherical particles) are formed depending on the heating temperature and the concentration of the reducing agent. In the present invention, gold or silver ion is used to form nanoparticles using a reducing agent. Specifically, a solution of HAuCl₄ is used as the gold ion, a solution of AgNO₃ is used as the silver ion, and sodium citrate is used as the reducing agent.

Nanoparticles formed according to the above-described method exhibit inherent colors. Generally, gold nanoparticles exhibit red color such as wine color, and silver nanoparticles have clear yellow color. If gold ion and silver ion are mixed and reduced simultaneously, an intermediate color between red color and yellow color (i.e., orange color) is shown. In this case, various colors can be developed by controlling the mixing ratio between the gold ion and the silver ion, since the more the silver ion is, the stronger the yellow color becomes, the more the gold ion is, the stronger the red color becomes.

Also, the present invention provides gold nanoshell particles exhibiting blue color. The gold nanoshell particles are obtained by cutting silver from the surface of silver nanoparticles by oxidation while reducing gold ions. As a result, empty spherical shells formed from gold particles alone are prepared, which have blue color. In this case, more various colors can be developed by controlling the shell size. The shell size can be controlled by changing the size of silver nanoparticles as template. Additionally, gold nanoparticles exhibiting blue color can be prepared in various forms, including nanorods, nanocubes, nanoprisms or the like.

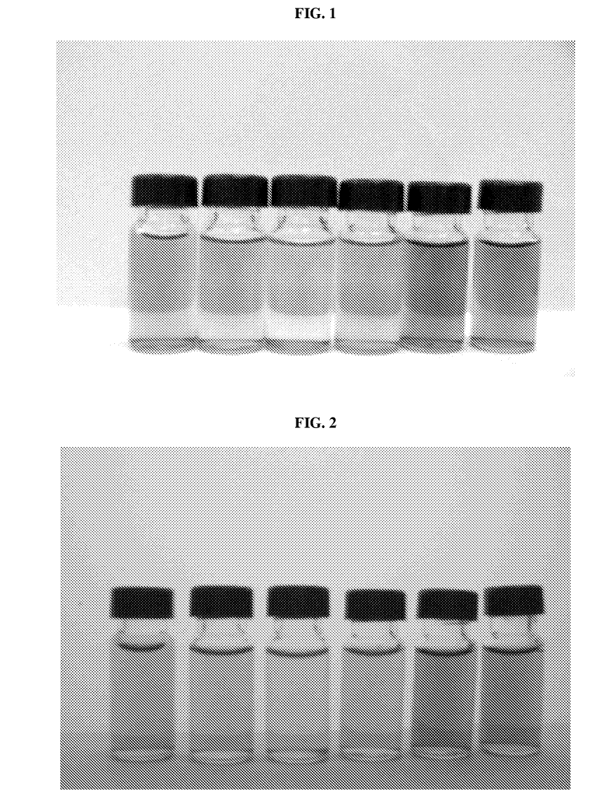

Nanoparticles exhibiting various colors having an absorption spectrum corresponding to the whole visible region can be prepared by mixing the above-prepared gold or silver nanoparticles exhibiting red color, yellow color and blue color at a suitable mixing ratio. Namely, a green color between yellow color and blue color can be developed by mixing nanoparticles exhibiting yellow color with nanoparticles exhibiting blue color, and a violet color between red color and blue color can be developed by mixing nanoparticles exhibiting red color with nanoparticles exhibiting blue color. Additionally, an orange color between red color and yellow color can be developed by mixing nanoparticles exhibiting red color with nanoparticles exhibiting yellow color. For example, as shown in FIG. 1, green color can be developed by mixing silver nanosolution exhibiting yellow color with gold nanoshell solution exhibiting blue color at a ratio of 7:3, and violet color can be developed by mixing gold nano-solution exhibiting red color with gold nanoshell solution exhibiting blue color at a ratio of 7:3. As a result, all colors in the visible region can be developed by combining three-color gold or silver nanoparticles with each other.

Since the gold/silver nanoparticle mixture according to the present invention is hydrophilic in nature, the mixture can be very well miscible with an aqueous solution. Accordingly, it is possible to provide novel pigments that can impart various colors to cosmetic compositions using a characteristic absorption spectrum and water-soluble property of the metal nanoparticles.

In the present invention, general lotions containing moisturizing agents, alcohols, purified water, and the like, are used to provide lotions comprising the pigment of the present invention. Cosmetics exhibiting inherent color are prepared by adding a suitable amount of nanoparticle solution exhibiting various colors to the lotions and mixing them. A lotion having desired color or chromaticity can be prepared by controlling the mixing ratio of the nanoparticle solutions. FIG. 2 shows color lotions exhibiting various colors, prepared by adding pure gold or silver nanoparticle composition or a mixture thereof to conventional lotions.

In particular, since the color lotions use gold and silver as pigments, the degeneration or discoloration of the pigment hardly occurs. Additionally, the color lotions can be maintained in a stable condition for a long period of time without adversely affecting the characteristic of the lotions.

In the present invention, the amount of the pigment composition added to a cosmetic composition is similar to the amount of a pigment which is conventionally used in the art. For example, the pigment composition of the present invention is preferably added in amounts of 0.0001 to 50 parts by weight, and more preferably 0.001 to 10 parts by weight, based on 100 parts by weight of the cosmetic composition.

### EXAMPLES

Hereinafter, the present invention will be described in detail with reference to examples. It is to be understood, however, that these examples are for illustrative purposes only and are not to be construed to limit the scope of the present invention.

### Example 1

### Preparation of Old Nanoparticles and Silver Nanoparticles

Each of 120 mg of gold ion (HAuCl₄) and 120 mg of silver ion (AgNO₃) was dissolved in 250 ml of water, and the resulting solutions were heated to 100° C. To each of the solutions, a solution containing 500 mg of trisodium citrate was added and the resulting solutions were reacted to prepare gold nanoparticle colloid exhibiting red color and silver nanoparticle colloid exhibiting yellow color, respectively.

### Example 2

### Preparation of Nanoparticles Exhibiting Flame Color

250 ml of a solution having 500 mg of trisodium citrate dissolved therein was heated to about 100° C., and reacted with a mixture of 2.5 mg/ml of gold ion (HAuCl₄) and 2.5 mg/ml of silver ion (AgNO₃) to prepare gold-silver alloy nanoparticles exhibiting orange colors. In addition to the method as described above, metal nanoparticle colloids exhibiting orange color can also be prepared by mixing the red-color gold nanoparticles prepared in example 1 with the yellow-color silver nanoparticles prepared in example 1 at a given ratio.

### Example 3

### Preparation of Nanoparticles Exhibiting Blue Color

40 mg of silver ion (AgNO₃), 1 g of PVP (polyvinylpyrrolidone) and 7.5 ml of EG (ethylene glycol) were mixed with each other, and the resulting solution was heated at 120° C. for 4 hrs and then cooled. The cooled solution was diluted 50-fold in 50 ml of trisodium citrate (0.4 mol), heated at 100° C. for 10 min, and then reacted with gold ion (HAuCl₄) which was being added in portions, thus preparing gold nanoshells exhibiting blue color.

In addition to the gold nanoshells as described above, gold nanoparticles exhibiting blue color can also be prepared in the form of gold nanorods, gold nanocubes, gold nanoprisms or the like. Gold nanorods can be prepared using surfactants such as CTAB upon the preparation of gold nanoparticles, and the absorption spectrum thereof can be controlled by adjusting the aspect ratio thereof. Nanocubes and nanoprisms having various absorption spectra can be prepared by adjusting their thickness and width using surfactants and photoreaction.

### Example 4

### Preparation of Lotion

The aqueous nanoparticle solutions (0.25 mg/ml) prepared in examples 1 to 3 were added to a lotion at various ratios (the volume ratio of nanoparticle aqueous solution:lotion=1:10-1:1) to a total volume of 10 ml, and sufficiently mixed to prepare water soluble cosmetics exhibiting various colors (see FIG. 2). As a result, as shown in FIG. 2, various colors of lotion could be developed by diversely adjusting the mixing ratio of gold or silver nanoparticles.

### Example 5

### Preparation of Lipstick

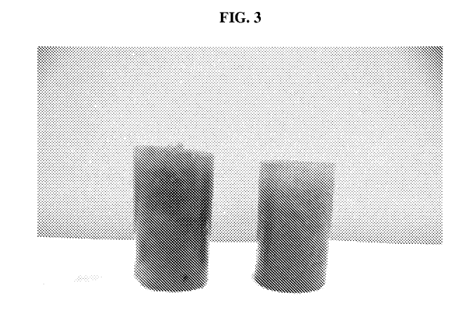

1 ml of gold nanoparticle solution (0.25 mg/ml) prepared in example 1 was mixed with 4 ml of transparent lipstick raw materials, and the resulting mixture was heated, cooled and then molded to prepare red lipstick (see FIG. 3). As a result, as shown in FIG. 3, it could be observed that the red color of the lipstick was clearly exhibited.

Lotions or lipsticks comprising the pigment composition of the present invention are completely free of any toxicity because these contain the pigment free of any metal harmful to the human body. Also, these are beneficial to health because these contain gold or silver.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

### INDUSTRIAL APPLICABILITY

As described above in detail, according to the present invention, it is possible to prepare pigments exhibiting various colors in the visible spectrum can be prepared using gold or silver nanoparticles, and a cosmetic pigment composition, which exhibits various colors, has no occurrence of particle settlement or cohesion therein and maintain its colors for a long period of time, can be prepared by mixing the pigments with each other in various ratios. Also, unlike the prior metal pigments, the pigment of the present invention is not harmful to the human body, has no side effects, and contains gold or silver beneficial to health. Thus, the inventive pigment can be used as a functional material in various applications.

1. A cosmetic pigment composition exhibiting the colors of the visible light spectrum, the composition comprising an effective amount of nanoparticles or a mixture of two or more nanoparticles selected from the group consisting of: (a) gold nanoparticles exhibiting red color; (b) silver nanoparticles exhibiting yellow color; (c) gold-silver alloy nanoparticles exhibiting orange color; and (d) gold nanoparticles exhibiting blue color.

2. The cosmetic pigment composition according to claim 1, wherein the nanoparticles are in the form of shapes selected from the group consisting of nanospheres, nanorods, nanoshells, nanocubes and nanoprisms.

3. The cosmetic pigment composition according to claim 1, wherein the gold nanoparticles exhibiting red color and the silver nanoparticles exhibiting yellow colors are in the form of nanospheres respectively, and the gold nanoparticles exhibiting blue color are in the form of shapes selected from the group consisting of nanoshells, nanorods, nanocubes and nanoprisms.

4. The cosmetic pigment composition according to claim 1, wherein the nanoparticle mixture is selected from the group consisting of: (a) a mixture in which gold nanoparticles exhibiting red color and silver nanoparticles exhibiting yellow color are mixed with each other at various ratios; (b) a mixture in which silver nanoparticles exhibiting yellow color and gold nanoparticles exhibiting blue color are mixed with each other at various ratios; (c) a mixture in which gold nanoparticles exhibiting red color and gold nanoparticles exhibiting blue color are mixed with each other at various ratios; (d) a mixture in which nanoparticles exhibiting three colors of red color, yellow color and blue color are mixed with each other at various ratios.

5. The cosmetic pigment composition according to claim 1, wherein the gold nanoparticles exhibiting red color are prepared through a method comprising the steps of:
(a) refluxing a solution of HAuCl₄ at about 100° C.;
(b) adding a reducing agent to the refluxed solution, and then heating the solution so as to react with the reducing agent; and
(c) cooling the reaction solution to room temperature and filtering the cooled solution.

6. The cosmetic pigment composition according to claim 1, wherein the silver nanoparticles exhibiting yellow color are prepared through a method comprising the steps of:
(a) refluxing a solution of AgNO₃ at about 100° C.;
(b) adding a reducing agent to the refluxed solution, and heating the solution so as to react with the reducing agent; and
(c) cooling the reaction solution to room temperature and filtering the cooled solution.

7. The cosmetic pigment composition according to claim 1, wherein the gold nanoparticles exhibiting blue color are prepared through a method comprising the steps of:
(a) mixing AgNO₃, PVP (polyvinylpyrrolidone) and EG (ethylene glycol), heating and reacting the mixture at about 120° C., and then cooling the reaction solution;
(b) adding a reducing agent to the cooled solution, heating the solution at about 100° C., and then allowing the heated solution to react with gold ion (HAuCl₄) which is being added in portions; and
(c) cooling the reaction solution to room temperature and filtering the cooled solution.

8. The cosmetic pigment composition according to claim 1, wherein the gold-silver alloy nanoparticles exhibiting orange color are prepared through a method comprising the steps of:
(a) heating a reducing agent solution, adding a mixture of gold ion (HAuCl₄) and silver ion (AgNO₃) to the heated reducing agent solution, and allowing the mixture solution to react; and
(b) cooling the reaction solution to room temperature and filtering the cooled solution.

9. A color cosmetics comprising the pigment composition of claim 1.

10. The color cosmetics according to claim 9, which have a formulation selected from the group consisting of twin-cake, emulsion foundation, make-up base, skin cover, eye shadow, face powder, a lipstick, mascara, nail-lacquer and eyebrow pencil.

11. A color lotion comprising the pigment composition of claim 1.

12. The color lotion according to claim 11, which has a formulation selected from the group consisting of skin softeners, skin lotions and essences.
